(19) United States
(12) Patent Application Publication
(10) Pub. No.: US 2018/0000365 A1
(43) Pub. Date: Jan. 4, 2018

(54) DETECTION DEVICE AND PROCESSING APPARATUS

(71) Applicant: Kabushiki Kaisha Toshiba, Minato-ku (JP)

(72) Inventors: Toshiya YONEHARA, Kawasaki (JP); Tomoaki SAWABE, Taito (JP); Akio AMANO, Machida (JP)

(73) Assignee: Kabushiki Kaisha Toshiba, Minato-ku (JP)

(21) Appl. No.: 15/705,964

(22) Filed: Sep. 15, 2017

Related U.S. Application Data
(63) Continuation of application No. PCT/JP2015/061693, filed on Apr. 16, 2015.

(51) Int. Cl. A61B 5/024; A61B 5/00

(52) U.S. Cl. CPC A61B 5/02427; A61B 5/6803

(57) ABSTRACT

According to one embodiment, a detection device includes a substrate, a light detector, a light emitter. The substrate is light-transmissive. The light emitter is provided between the substrate and the light detector. The light emitter includes a first electrode, a light-emitting layer, and a plurality of second electrodes. The first electrode is provided between the light detector and the substrate. The first electrode is light-transmissive. The light-emitting layer is provided between the light detector and the first electrode. The second electrodes are provided between the light detector and the light-emitting layer.

### CROSS-REFERENCE TO RELATED APPLICATIONS

This is a continuation application of International Application PCT/JP2015/061693, filed on Apr. 16, 2015; the entire contents of which are incorporated herein by reference.

### FIELD

Embodiments described herein relate generally to a detection device and a processing apparatus.

### BACKGROUND

There is a detection device in which light is radiated from a light emitter and irradiated onto a detection object, and the light that is reflected by the detection object is detected. It is desirable for the detection device to be small.

### BRIEF DESCRIPTION OF THE DRAWINGS

FIG. 1A and FIG. 1B are schematic views illustrating an example of a detection device according to a first embodiment;

FIG. 2A and FIG. 2B are schematic cross-sectional views illustrating examples of optical paths of detection devices;

FIG. 3 is a schematic cross-sectional view illustrating another example of the detection device according to the first embodiment;

FIG. 4A to FIG. 4E illustrate simulation results of the detection device;

FIG. 5 is a schematic view illustrating the relationship between the efficiency and the detection position of the light;

FIG. 6A to FIG. 6E, FIG. 7A to FIG. 7E, and FIG. 8A to FIG. 8E illustrate other simulation results of detection devices;

FIG. 9A and FIG. 9B are schematic views illustrating another example of the detection device according to the first embodiment;

FIG. 10 to FIG. 12 are schematic plan views illustrating other examples of the detection device according to the first embodiment;

FIG. 13 is a schematic cross-sectional view illustrating an example of a detection device according to a second embodiment;

FIG. 14 is a schematic cross-sectional view illustrating another example of the detection device according to the second embodiment;

FIG. 15 is a schematic cross-sectional view illustrating an example of a detection device according to a third embodiment;

FIG. 16 is a schematic cross-sectional view illustrating another example of the detection device according to the third embodiment;

FIG. 17 is a schematic cross-sectional view illustrating an example of a detection device according to a fourth embodiment;

FIG. 18 is a schematic cross-sectional view illustrating another example of the detection device according to the fourth embodiment;

FIG. 19 is a schematic cross-sectional view illustrating another example of the detection device according to the fourth embodiment;

FIG. 20 is a schematic cross-sectional view illustrating another example of the detection device according to the fourth embodiment;

FIG. 21 and FIG. 22 are schematic views illustrating examples of a processing apparatus including the detection device according to the embodiment;

FIG. 23A to FIG. 26B are schematic views illustrating a pulse wave being measured using the detection device according to the embodiment;

FIG. 27A and FIG. 27B are schematic views illustrating processing apparatuses including the detection device according to the embodiment;

FIG. 28A to FIG. 28E are schematic views illustrating applications of processing apparatuses including the detection device according to the embodiment; and

FIG. 29 is a schematic view illustrating a system including the processing apparatuses illustrated in FIGS. 28A to 28E.

### DETAILED DESCRIPTION

According to one embodiment, a detection device includes a substrate, a light detector, a light emitter. The substrate is light-transmissive. The light emitter is provided between the substrate and the light detector. The light emitter includes a first electrode, a light-emitting layer, and a plurality of second electrodes. The first electrode is provided between the light detector and the substrate. The first electrode is light-transmissive. The light-emitting layer is provided between the light detector and the first electrode. The second electrodes are provided between the light detector and the light-emitting layer.

Embodiments of the invention will now be described with reference to the drawings.

The drawings are schematic or conceptual; and the relationships between the thicknesses and widths of portions, the proportions of sizes between portions, etc., are not necessarily the same as the actual values thereof. The dimensions and/or the proportions may be illustrated differently between the drawings, even in the case where the same portion is illustrated.

In the drawings and the specification of the application, components similar to those described thereinabove are marked with like reference numerals, and a detailed description is omitted as appropriate.

### First Embodiment

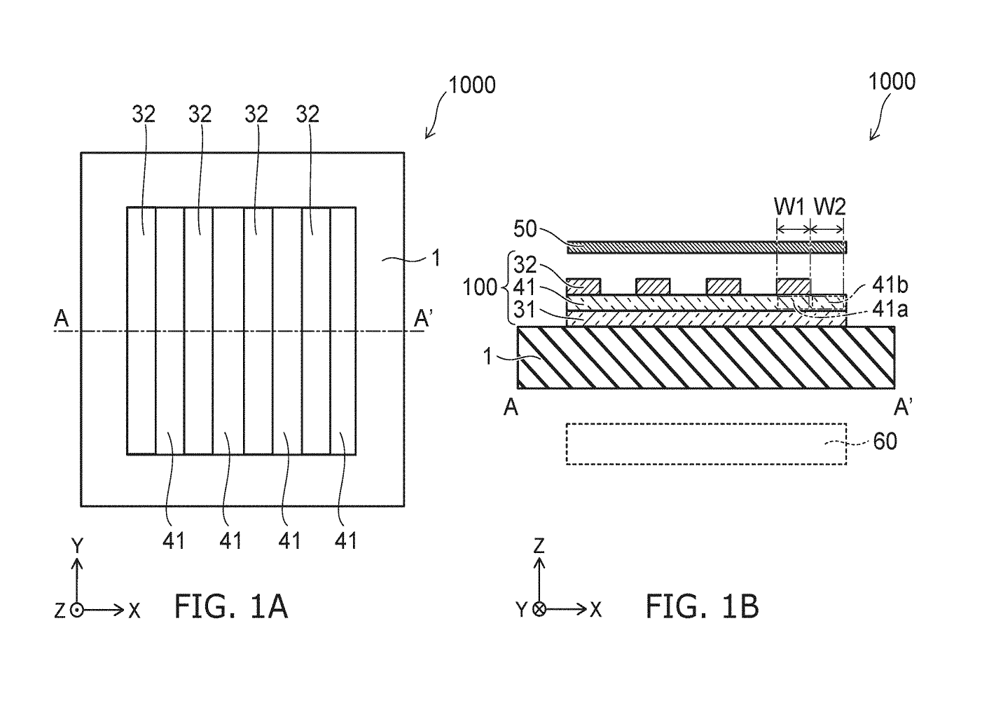

FIG. 1A and FIG. 1B are schematic views illustrating an example of a detection device according to a first embodiment. FIG. 1A is a schematic plan view; and FIG. 1B is a schematic cross-sectional view illustrating an A-A′ cross section of FIG. 1A. A light detector 50 is not illustrated in FIG. 1A.

A detection device 1000 includes a substrate 1, the light detector 50, and a light emitter 100 as illustrated in FIG. 1B. The light emitter 100 includes a first electrode 31, a light-emitting layer 41, and multiple second electrodes 32.

A direction from the substrate 1 toward the light detector 50 is taken as a first direction. The first direction is, for example, a Z-direction illustrated in FIG. 1A and FIG. 1B. Two directions perpendicular to each other and perpendicular to the first direction are taken respectively as a second direction and a third direction. For example, the second direction is an X-direction; and the third direction is a Y-direction.

The first electrode 31 is provided between at least a portion of the substrate 1 and at least a portion of the light detector 50. The light-emitting layer 41 is provided between the first electrode 31 and at least a portion of the light detector 50. The multiple second electrodes 32 are provided between the light-emitting layer 41 and the light detector 50. For example, the light detector 50 is provided to be separated from the multiple second electrodes 32 in the first direction.

In the example illustrated in FIG. 1A and FIG. 1B, the multiple second electrodes 32 are arranged in the second direction; and each of the second electrodes 32 extends in the third direction. The light-emitting layer 41 includes multiple light-emitting regions 41a and multiple non-light-emitting regions 41b. The light-emitting regions 41a are positioned respectively between the first electrode 31 and the second electrodes 32 in the first direction. The non-light-emitting regions 41b are not positioned respectively between the first electrode 31 and the second electrodes 32 in the first direction. For example, the light-emitting regions 41a and the non-light-emitting regions 41b are provided alternately in the second direction.

The light detector 50 is arranged with at least the light-emitting regions 41a in the first direction. More desirably, the light detector 50 is arranged with both the light-emitting regions 41a and the non-light-emitting regions 41b in the first direction. By the light detector 50 being arranged with the multiple light-emitting regions 41a and the multiple non-light-emitting regions 41b in the first direction, the amount of the light incident on the light detector 50 can be increased.

When carriers are injected into the light-emitting layer 41 from the first electrode 31 and the second electrodes 32, light is radiated mainly from the light-emitting regions 41a. The noise is smaller for the light radiated from a light-emitting element using a light-emitting layer including an organic substance than for the light radiated from a light-emitting element using a light-emitting layer including an inorganic compound. Therefore, the light that is radiated from the light-emitting element using the light-emitting layer including the organic substance is suited to, for example, applications that detect a detection object such as a pulse wave, etc., in which the signal that is output is faint.

The substrate 1 and the first electrode 31 transmit the light radiated from the light-emitting layer 41. The substrate 1 and the first electrode 31 are light-transmissive. The second electrodes 32 are light-reflective. The reflectance of the second electrodes 32 is higher than the reflectance of the first electrode 31 and higher than the reflectance of the substrate 1. The second electrodes 32 reflect the light radiated from the light-emitting layer 41 toward the substrate 1. Therefore, the amount of the light directly incident on the light detector 50 from the light-emitting layer 41 is reduced; and the detection sensitivity can be increased.

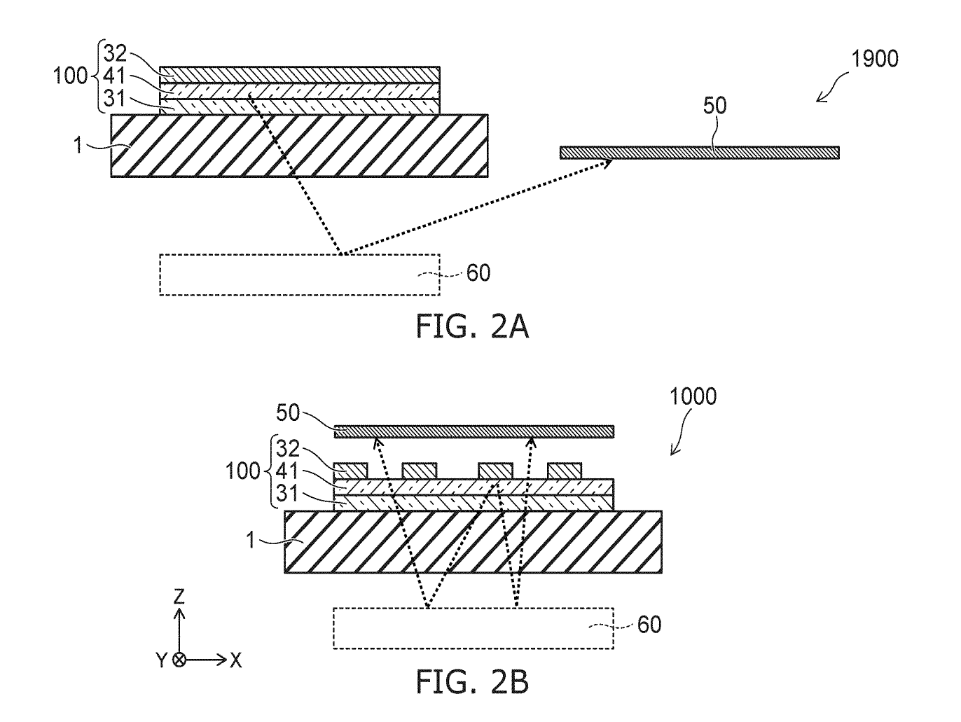

FIG. 2A and FIG. 2B are schematic cross-sectional views illustrating examples of optical paths of detection devices. FIG. 2A illustrates an example of the optical path of a detection device 1900 according to a reference example. FIG. 2B illustrates an example of the optical path of the detection device 1000 according to the embodiment.

In the detection device 1900, the light detector 50 is arranged in the second direction with the substrate 1. The light that is radiated from the light emitter 100 is reflected by a detection object 60, travels in the second direction, and is incident on the light detector 50.

On the other hand, in the detection device 1000, the light emitter 100 and the light detector 50 overlap in the first direction. The light emitter 100 is positioned between the detection object 60 and the light detector 50. The light that is radiated from the light-emitting layer 41 is reflected by the detection object 60. The light that is reflected passes through a gap between the second electrodes 32 and is incident on the light detector 50.

The optical path of the light radiated from the light-emitting layer 41 until being incident on the light detector 50 can be shortened because the multiple second electrodes 32 are provided between the light-emitting layer 41 and the light detector 50 and the reflected light from the detection object 60 passes through the gap between the second electrodes 32. As a result, it is possible to downsize the detection device while suppressing the decrease of the detection sensitivity.

Examples of the components will now be described.

The substrate 1 includes, for example, glass. The refractive index of the substrate 1 is, for example, not less than 1.4 and not more than 2.2. A thickness T1 along the first direction of the substrate 1 is, for example, 0.05 to 2.0 mm.

The second electrode 32 includes, for example, at least one of aluminum, silver, or gold. The second electrode 32 includes, for example, an alloy of magnesium and silver.

The first electrode 31 includes, for example, ITO (Indium Tin Oxide). The first electrode 31 may include, for example, a conductive polymer such as PEDOT:PSS, etc. The first electrode 31 may include a metal such as aluminum, silver, etc. In the case where the first electrode 31 includes a metal, it is favorable for the thickness of the first electrode 31 to be 5 to 20 nm.

The light-emitting layer 41 includes, for example, at least one of Alq₃ (tris(8-hydroxyquinolinolato)aluminum), F8BT (poly(9,9-dioctylfluorene-co-benzothiadiazole), or PPV (polyparaphenylene vinylene).

Or, the light-emitting layer 41 may include a mixed material containing a host material and a dopant. The host material includes, for example, at least one of CBP (4,4′-N,N′-bis dicarbazolyl-biphenyl), BCP (2,9-dimethyl-4,7diphenyl-1,10-phenanthroline), TPD (2,9-dimethyl-4,7diphenyl-1,10-phenanthroline), PVK (polyvinyl carbazole), or PPT (poly(3-phenylthiophene)). The dopant material includes, for example, at least one of Flrpic (iridium(III)-bis(4,6-di-fluorophenyl)-pyridinate-N,C2′-picolinate), Ir(ppy)₃ (tris(2-phenylpyridine)iridium), or Flr6 (bis(2,4-difluorophenylpyridinato)-tetrakis(1-pyrazolyl)borate-iridium(III)).

The light that is radiated from the light-emitting layer 41 is, for example, visible light. The light that is radiated from the light-emitting layer 41 is, for example, one of red, orange, yellow, green, or blue light or a combination of such light. The light that is radiated from the light-emitting layer 41 may be ultraviolet light or infrared light.

In a plane perpendicular to the first direction, the configuration of the first electrode 31 and the configuration of the light-emitting layer 41 are, for example, polygons (of which the corners may be curves) or circles (including flattened circles). These configurations are arbitrary. In a plane perpendicular to the first direction, the configuration of each of the second electrodes 32 is, for example, a polygon (of which the corners may be curves) or a circle (including a flattened circle). The configuration of each of the second electrodes 32 is arbitrary.

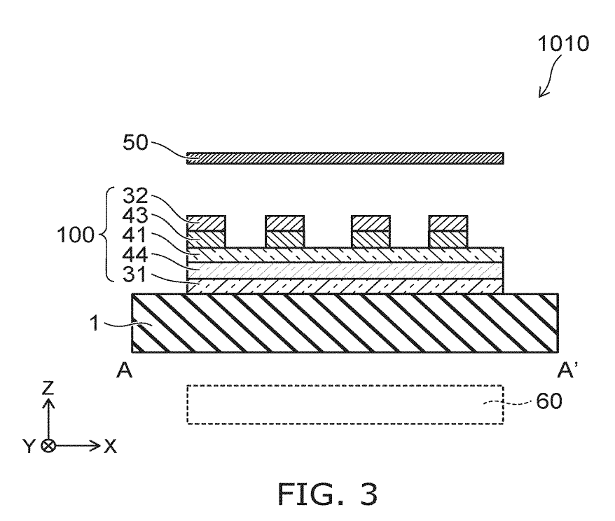

FIG. 3 is a schematic cross-sectional view illustrating another example of the detection device according to the first embodiment. In the detection device 1010 illustrated in FIG. 3, the light emitter 100 may further include a third layer 43 and a fourth layer 44. For example, the third layer 43 is multiply provided in the second direction; and the third layers 43 are provided respectively between the light-emitting layer 41 and the second electrodes 32. Or, the third layer 43 may be provided on the entire surface of the light-emitting layer 41. The fourth layer is provided between the first electrode 31 and the light-emitting layer 41.

The third layer 43 functions as, for example, a carrier injection layer. In such a case, the third layer 43 may function as an electron injection layer. The third layer 43 may function as a carrier transport layer. In such a case, the third layer 43 may function as an electron transport layer. The third layer 43 may include a layer functioning as a carrier injection layer and a layer functioning as a carrier transport layer.

The third layer 43 includes, for example, at least one of Alq₃, BAlq, POPy₂, Bphen, or 3TPYMB. In the case where the third layer 43 includes at least one of these materials, the third layer 43 functions as an electron transport layer.

Or, the third layer 43 includes, for example, at least one of LiF, CsF, Ba, or Ca. In the case where the third layer 43 includes at least one of these materials, the third layer 43 functions as an electron injection layer.

The fourth layer 44 functions as, for example, a carrier injection layer. In such a case, the fourth layer 44 may function as a hole injection layer. The fourth layer 44 may function as a carrier transport layer. In such a case, the fourth layer 44 may function as a hole transport layer. The fourth layer 44 may include a layer functioning as a carrier injection layer and a layer functioning as a carrier transport layer.

The fourth layer 44 includes, for example, at least one of α-NPD, TAPC, m-MTDATA, TPD, or TCTA. In the case where the fourth layer 44 includes at least one of these materials, the fourth layer 44 functions as a hole transport layer.

Or, the fourth layer 44 includes, for example, at least one of PEDPOT:PPS, CuPc, or MoO₃. In the case where the fourth layer 44 includes at least one of these materials, the fourth layer 44 functions as a hole injection layer.

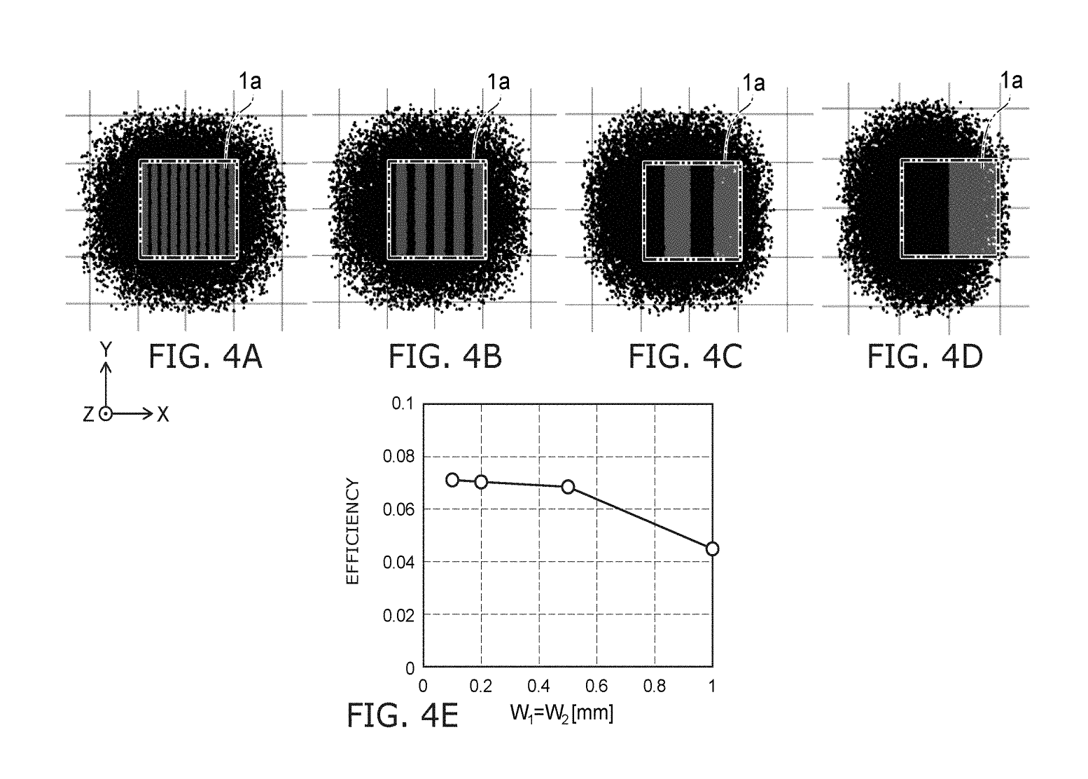

FIG. 4A to FIG. 4E illustrate simulation results of the detection device. FIG. 4A to FIG. 4D are schematic plan views illustrating distributions of the light and structures of the light emitter used in the simulations. FIG. 4A to FIG. 4C illustrate the structures of the light emitter included in the detection device according to the first embodiment; and FIG. 4D illustrates the structure of the light emitter included in a detection device according to a reference example.

In FIG. 4A to FIG. 4D, the distributions of the coordinates when the light radiated from the light-emitting regions 41a is reflected from the detection object 60 and reaches the surface of the substrate 1 on the first electrode 31 side are illustrated. In each of the figures, a region 1a illustrates the region where the first electrode 31 and the light-emitting layer 41 overlap in the first direction. The dots illustrated in gray illustrate the light incident on the regions overlapping the non-light-emitting regions 41b in the first direction in the surface of the substrate 1 on the first electrode 31 side.

FIG. 4E is a graph illustrating the change of the efficiency when a width W1 and a width W2 are changed. The efficiency illustrates the proportion (L0/L1) of a light amount L0 to a light amount L1, where the light amount L1 is radiated from the light-emitting regions 41a, and the light amount L0 passes through the substrate 1, is reflected by the detection object 60, and subsequently passes through the non-light-emitting regions 41b.

The width W1 is the length in the second direction of the light-emitting region 41a. The width W2 is the length in the second direction of the non-light-emitting region 41b. For example, the width W1 is equal to the length in the second direction of the second electrode 32. For example, the width W2 is equal to the distance in the second direction between the mutually-adjacent second electrodes 32.

In the simulation, the distance in the first direction between the substrate 1 and the detection object 60 is 0 mm; and the light that is emitted outside the substrate 1 is immediately incident on the detection object 60.

The other conditions are as follows. The thickness in the first direction of the substrate 1 is 0.7 mm. The length in the second direction and the length in the third direction of the light-emitting layer 41 are 2 mm. The size and configuration of the first electrode 31 are the same as the size and configuration of the light-emitting layer 41. The refractive index of the substrate 1 is 1.5. The light source is isotropic. The thicknesses in the first direction of the first electrode 31 and the light-emitting layer 41 each are, for example, 10 to 100 nm. Accordingly, because the first electrode 31 and the light-emitting layer 41 are sufficiently thinner than the substrate 1, the position in the first direction of the light source is taken to be the portion where the substrate 1 contacts the first electrode 31.

In the detection device illustrated in FIG. 4A, the width W1 and the width W2 are 0.1 mm. In the detection device illustrated in FIG. 4B, the width W1 and the width W2 are 0.2 mm. In the detection device illustrated in FIG. 4C, the width W1 and the width W2 are 0.5 mm. In the detection device illustrated in FIG. 4D, the width W1 and the width W2 are 1.0 mm.

It can be seen in the graph illustrated in FIG. 4E that the efficiency is increased more for the configurations illustrated in FIGS. 4A to 4C than for the configuration illustrated in FIG. 4D. Accordingly, it can be seen that the efficiency can be increased by subdividing the second electrodes 32 into a plurality. Further, it can be seen that the efficiency increases as the width W1 and the width W2 decrease and the second electrodes 32 are subdivided more.

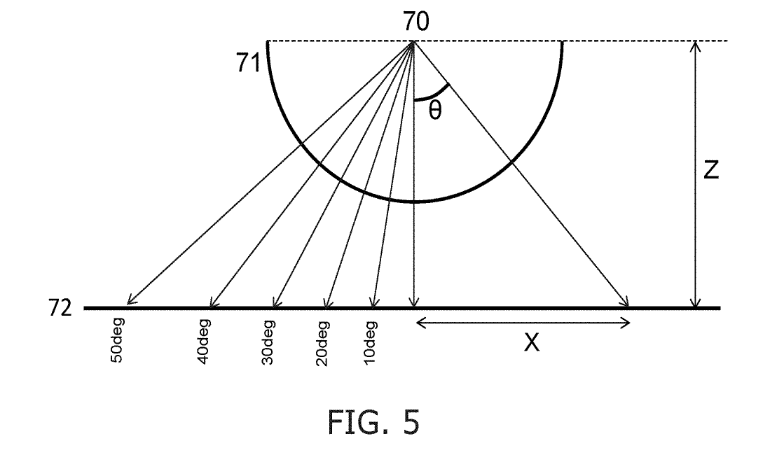

This aspect will now be described using FIG. 5. FIG. 5 is a schematic view illustrating the relationship between the efficiency and the detection position of the light. In the example illustrated in FIG. 5, the light is radiated isotropically from a light source 70. In the example, the amount of the light passing per unit surface area of a curved surface 71 is constant at all locations. Conversely, at a plane 72, the amount of the light incident per unit surface area decreases as the distance from the light source 70 increases.

In FIG. 5, the minimum distance between the light source 70 and the plane 72 is taken as Z; and the radiation angle of the light from the light source 70 to the plane 72 is taken as B. In such a case, a position X where the light radiated from the light source 70 is incident on the plane 72 is represented by the following Formula (1).

X=Z×tan θ (1)

The following Formula (2) is obtained by differentiating Formula (1) by θ.

$$\frac{d X}{d 0} = \frac{Z}{{( cos 0 )}^{2}} \qquad ( 2 )$$

From Formula (2), it can be seen that X increases as the irradiation angle θ increases. Therefore, it can be seen that the amount of the light incident per unit surface area of the plane 72 decreases away from the light source 70.

In the case where the second electrode 32 is subdivided into a plurality, the light passes through the gap between the second electrodes 32 and is incident on the light detector 50. In other words, the minimum value of θ can be reduced for the light incident on the light detector 50. As the second electrodes 32 are subdivided further, the minimum value of θ also decreases; and the efficiency can be increased. These aspects match the simulation results illustrated in FIG. 4E.

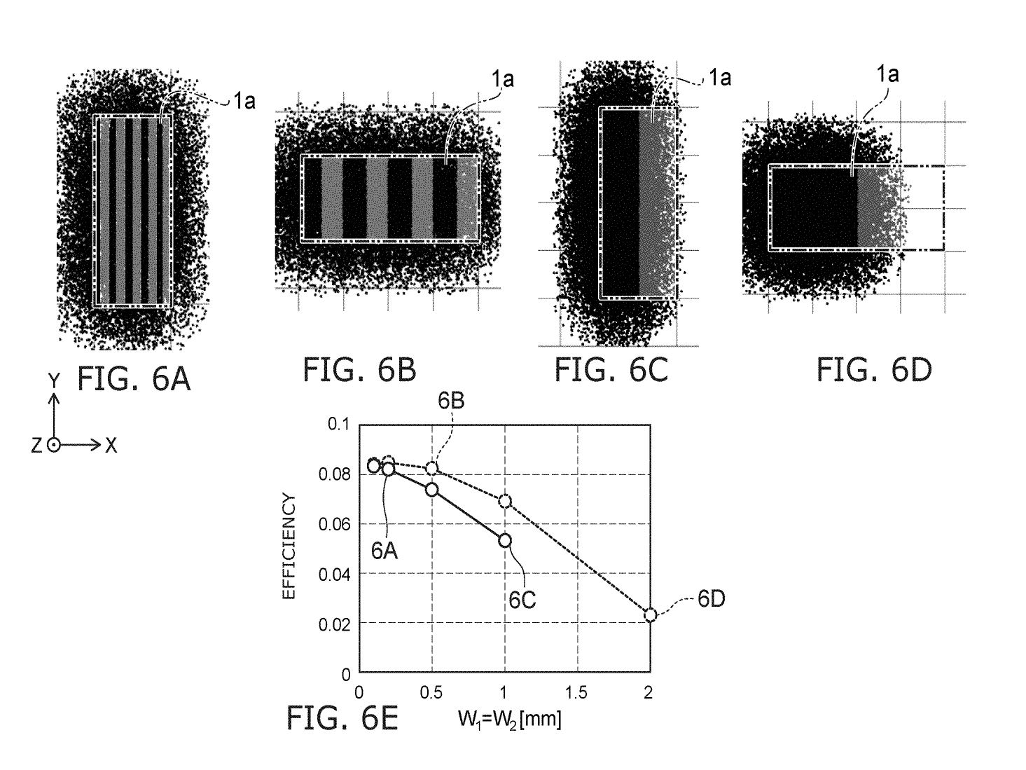

FIG. 6A to FIG. 6E, FIG. 7A to FIG. 7E, and FIG. 8A to FIG. 8E illustrate other simulation results of detection devices.

FIG. 6A to FIG. 6D, FIG. 7A to FIG. 7D, and FIG. 8A to FIG. 8D are schematic plan views illustrating the structure of the light emitter and the distribution of the light used in each simulation similarly to FIG. 4A to FIG. 4D. FIG. 6E, FIG. 7E, and FIG. 8E are graphs illustrating the change of the efficiency when the width W1 and the width W2 are changed.

FIG. 6A, FIG. 6B, FIG. 7A to FIG. 7C, and FIG. 8A to FIG. 8C illustrate the structures of the light emitters included in other detection devices according to the first embodiment. FIG. 6C, FIG. 6D, FIG. 7D, and FIG. 8D illustrate the structures of the light emitters included in other detection devices according to reference examples.

The conditions that relate to the thickness of the substrate 1, the refractive index of the substrate 1, and the light source are similar to the conditions used in the simulation illustrated in FIGS. 4A to 4E.

In the graph illustrated in FIG. 6E, the solid line illustrates the results in the case where the length in the second direction of the light-emitting layer 41 is 2 mm and the length in the third direction of the light-emitting layer 41 is 4 mm. The broken line illustrates the results in the case where the length in the second direction of the light-emitting layer 41 is 4 mm and the length in the third direction of the light-emitting layer 41 is 2 mm.

It can be seen from FIG. 6E that in each case, the efficiency increases as the width W1 and the width W2 become narrower. For the same widths W1 and W2, it can be seen that the efficiency is higher for the case where the length in the second direction of the light-emitting layer 41 is longer than the length in the third direction of the light-emitting layer 41 than for the case where the length in the third direction of the light-emitting layer 41 is longer than the length in the second direction of the light-emitting layer 41. This is because the second electrodes 32 are subdivided more in the case where the length in the second direction of the light-emitting layer 41 is longer than the length in the third direction of the light-emitting layer 41.

Comparing FIG. 6C and FIG. 6D, it can be seen that the efficiencies are different even for the same number of subdivided second electrodes 32. Specifically, in the case where the number of the second electrodes 32 is the same, the efficiency is higher for the detection device in which the multiple second electrodes 32 are arranged along the short-side directions of the first electrode 31 and the light-emitting layer 41 than for the detection device in which the multiple second electrodes 32 are arranged along the long-side directions.

FIG. 7A to FIG. 7E illustrate simulation results in the case where the lengths in the second direction and the third direction of the light-emitting layer 41 are 10 mm. From the results illustrated in FIG. 7E, it can be seen that the efficiency increases as the width W1 and the width W2 decrease and the second electrodes 32 are subdivided more.

FIG. 8A to FIG. 8E illustrate simulation results in the case where the length in the second direction of the light-emitting layer 41 is 4 mm and the length in the third direction of the light-emitting layer 41 is 2 mm. In FIGS. 8A to 8E, the distance in the first direction between the substrate 1 and the detection object 60 is set to 2 mm. From the results illustrated in FIG. 8E, it can be seen that the efficiency increases as the width W1 and the width W2 decrease as the second electrodes 32 are subdivided more.

In the detection device according to the embodiment as illustrated in FIGS. 4A to 4E, FIGS. 6A to 6E, FIGS. 7A to 7E, and FIGS. 8A to 8E, the second electrodes 32 are subdivided in the second direction; and the length in the third direction of the second electrode 32 is longer than the length in the second direction of the second electrode 32. It is possible to easily electrically connect each of the second electrodes 32 to the other interconnects by drawing out each of the second electrodes 32 outside the region overlapping the light-emitting layer 41 in the first direction by further extending each of the second electrodes 32 in the third direction. In other words, the detection device can be made more easily by employing such a configuration.

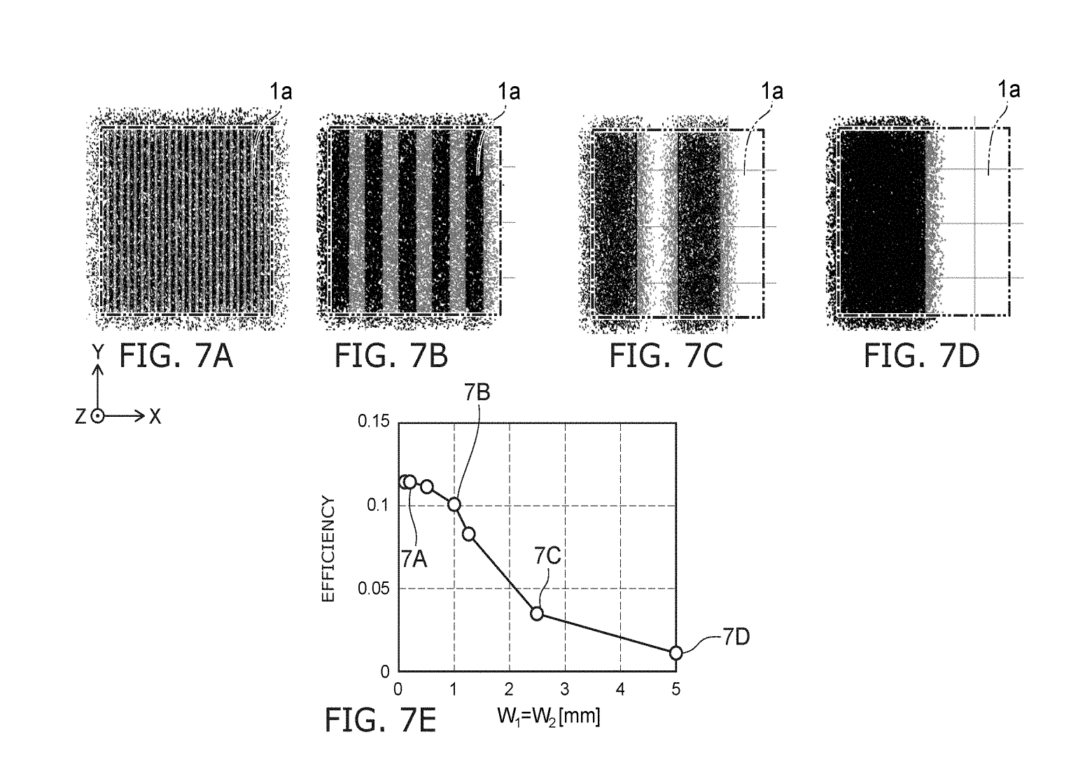

FIG. 9A and FIG. 9B are schematic views illustrating another example of the detection device according to the first embodiment. FIG. 9A is a schematic plan view; and FIG. 9B is a schematic cross-sectional view illustrating an A-A′ cross section of FIG. 9A. The light detector 50 is not illustrated in FIG. 9A. As illustrated in FIG. 9A, the configuration of the light-emitting layer 41 when viewed from the first direction is, for example, a circle. The detection device 1100 includes the multiple second electrodes 32 provided in annular configurations. The multiple second electrodes 32 are provided to be separated from each other.

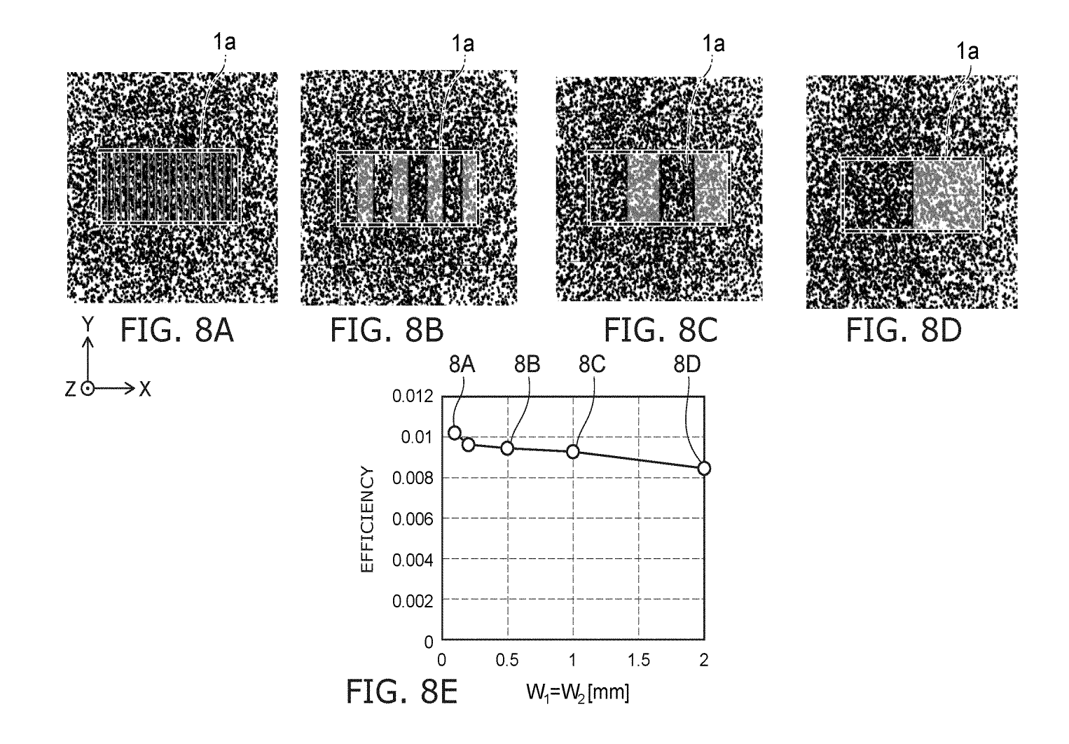

FIG. 10 to FIG. 12 are schematic plan views illustrating other examples of the detection device according to the first embodiment. The light detector 50 is not illustrated in FIG. 10 to FIG. 12. For example, the structures of the A-A′ cross sections of FIG. 10 to FIG. 12 are similar to FIG. 1B.

A detection device 1200 illustrated in FIG. 10 includes the multiple second electrodes 32. The multiple second electrodes 32 are arranged in the second direction and the third direction to be separated from each other.

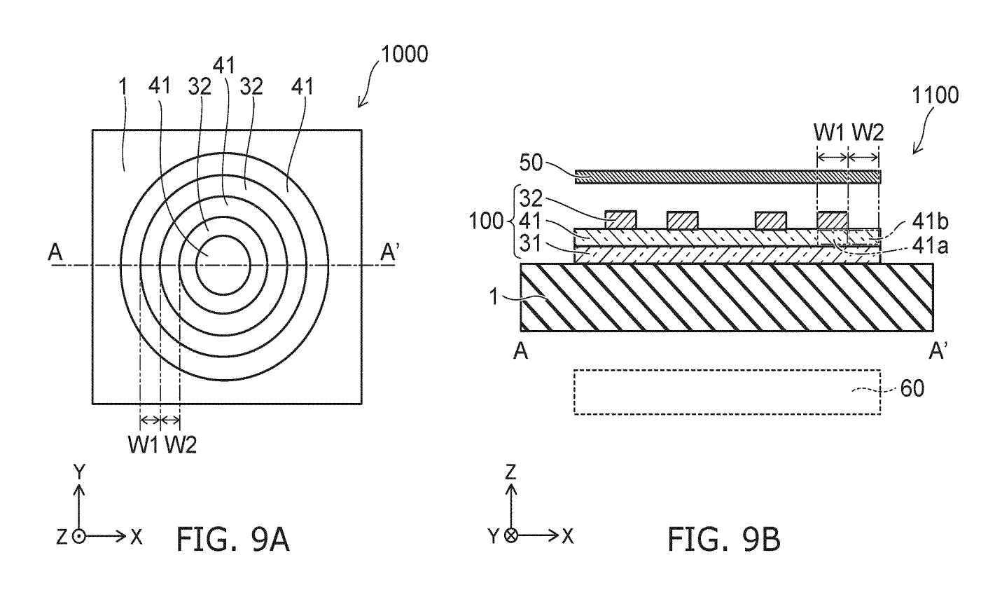

A detection device 1300 illustrated in FIG. 11 includes, for example, one second electrode 32. The second electrode 32 includes multiple first portions 32a. The multiple first portions 32a are arranged in the second direction to be separated from each other. The light that is reflected by the detection object 60 passes through the gaps in the second direction between the first portions 32a and is incident on the light detector 50. For example, the width W1 of the light-emitting region 41a is equal to the length in the second direction of the first portion 32a. For example, the width W2 of the non-light-emitting region 41b is equal to the distance in the second direction between the mutually-adjacent first portions 32a.

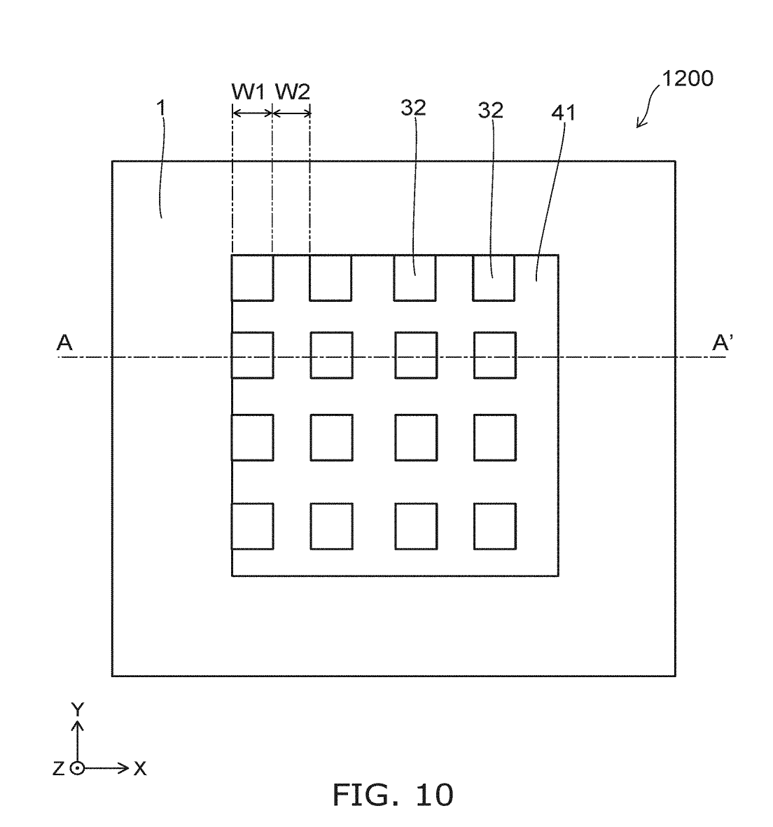

A detection device 1400 illustrated in FIG. 12 includes, for example, one second electrode 32. The second electrode 32 includes the multiple first portions 32a. The multiple first portions 32a are arranged to be separated from each other in the second direction and the third direction. The light that is reflected by the detection object 60 passes through the gap in the second direction and the gap in the third direction between the first portions 32a and is incident on the light detector 50.

The second electrode 32 includes a portion extending in the second direction and a portion extending in the third direction. For example, the width W1 of the light-emitting region 41a is equal to the length in the second direction of the portion extending in the third direction. The width W1 may be equal to the length in the third direction of the portion extending in the second direction. For example, the width W2 of the non-light-emitting region 41b is equal to the distance in the second direction between the first portions 32a. The width W2 may be equal to the distance in the third direction between the first portions 32a.

In the examples of the detection devices described above, the width W1 may be the same as or different from the width W2.

### Second Embodiment

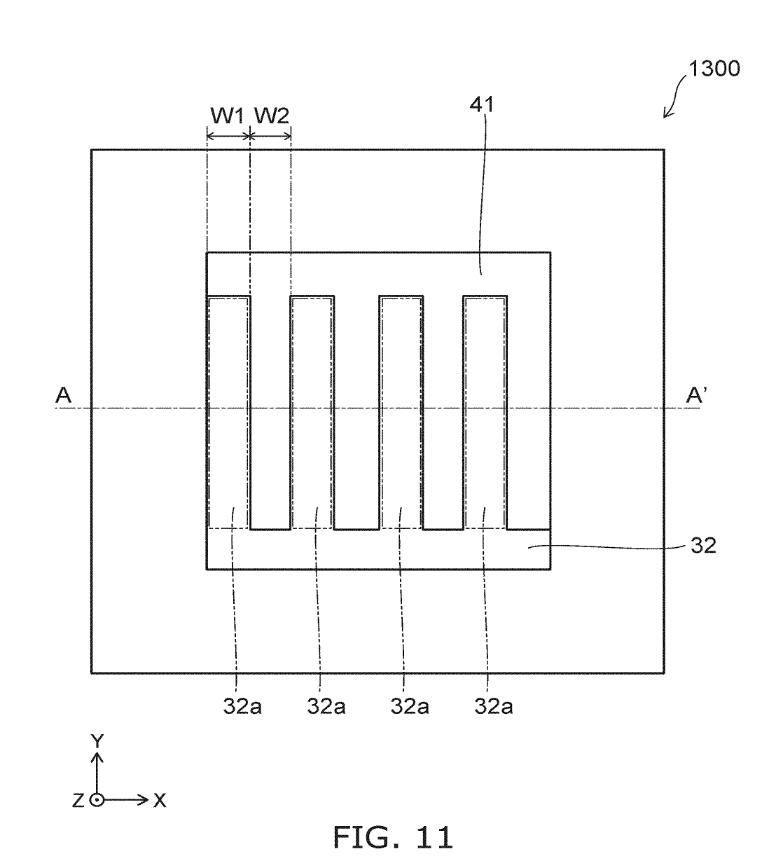

FIG. 13 is a schematic cross-sectional view illustrating an example of a detection device according to a second embodiment. As illustrated in FIG. 13, the detection device 2000 includes the substrate 1, the first electrode 31, the light-emitting layer 41, the multiple second electrodes 32, a fourth electrode 34, a photoelectric conversion layer 51, and a third electrode 33.

The photoelectric conversion layer 51 is provided between the third electrode 33 and the light-emitting layer 41. The fourth electrode 34 is provided between the photoelectric conversion layer 51 and the light-emitting layer 41. The fourth electrode 34 is light-transmissive. The multiple second electrodes 32 are provided between the fourth electrode 34 and the light-emitting layer 41.

For example, the multiple second electrodes 32 are arranged in the second direction. The structures illustrated in any of FIG. 9A to FIG. 12 also are employable as the structure of the second electrodes 32. For example, a portion of the fourth electrode 34 is provided between the second electrodes 32 in the second direction.

The injection barrier between the fourth electrode 34 and the light-emitting layer 41 is larger than the injection barrier between the light-emitting layer 41 and the second electrode 32. Therefore, the carriers are injected into the light-emitting layer 41 mainly from the first electrode 31 and the multiple second electrodes 32; and the light is radiated mainly from the light-emitting regions 41a positioned respectively between the first electrode 31 and the second electrodes 32.

In the case where the detection device 2000 includes the third layer 43 provided between the light-emitting layer 41 and the multiple second electrodes 32, the injection barrier between the fourth electrode 34 and the third layer 43 is larger than the injection barrier between the third layer 43 and the second electrode 32. Therefore, the carriers are injected into the light-emitting layer 41 mainly from the first electrode 31 and the multiple second electrodes 32; and the light is radiated mainly from the light-emitting regions 41a positioned respectively between the first electrode 31 and the second electrodes 32.

In the case where the third layer 43 that functions as an electron injection layer is provided in contact with the second electrodes 32 between the light-emitting layer 41 and the multiple second electrodes 32, the material that is included in the second electrodes 32 may be the same as the material included in the fourth electrode 34. Even in the case where the second electrodes 32 and the fourth electrode 34 include the same material, the injection amount of the electrons from the second electrodes 32 into the light-emitting layer 41 is higher than the injection amount of the electrons from the fourth electrode 34 into the light-emitting layer 41 because the third layer 43 is provided. Therefore, the light is radiated mainly from the light-emitting regions 41a positioned respectively between the first electrode 31 and the second electrodes 32.

The third electrode 33, the photoelectric conversion layer 51, and the fourth electrode 34 may function as light detectors. The light that is radiated from the light-emitting layer 41 is reflected by the detection object 60, passes through the gap between the second electrodes 32, and is incident on the photoelectric conversion layer 51. When the light is incident on the photoelectric conversion layer 51, a current flows between the third electrode 33 and the fourth electrode 34; therefore, the information that relates to the detection object 60 can be obtained by detecting the current.

The third electrode 33 includes, for example, at least one of aluminum, silver, or gold. The third electrode 33 includes, for example, an alloy of magnesium and silver.

The fourth electrode 34 includes, for example, ITO. The fourth electrode 34 may include a metal such as aluminum, silver, etc. In the case where the fourth electrode 34 includes a metal, it is favorable for the thickness in the first direction of the fourth electrode 34 to be 5 to 20 nm.

The photoelectric conversion layer 51 includes, for example, at least one of a porphyrin cobalt complex, a coumarin derivative, fullerene, a fullerene derivative, a fluorene compound, a pyrazole derivative, a quinacridone derivative, a perylene bisimide derivative, an oligothiophene derivative, a subphthalocyanine derivative, a rhodamine compound, a ketocyanine derivative, a phthalocyanine derivative, a squarylium derivative, or a subnaphthalocyanine derivative.

For example, the porphyrin cobalt complex, the coumarin derivative, the fullerene, the derivative of fullerene, the fluorene compound, and the pyrazole derivative selectively absorb blue light.

For example, the quinacridone derivative, the perylene bisimide derivative, the oligothiophene derivative, the subphthalocyanine derivative, the rhodamine compound, and the ketocyanine derivative selectively absorb green light.

For example, the phthalocyanine derivative, the squarylium derivative, and the subnaphthalocyanine derivative selectively absorb red light.

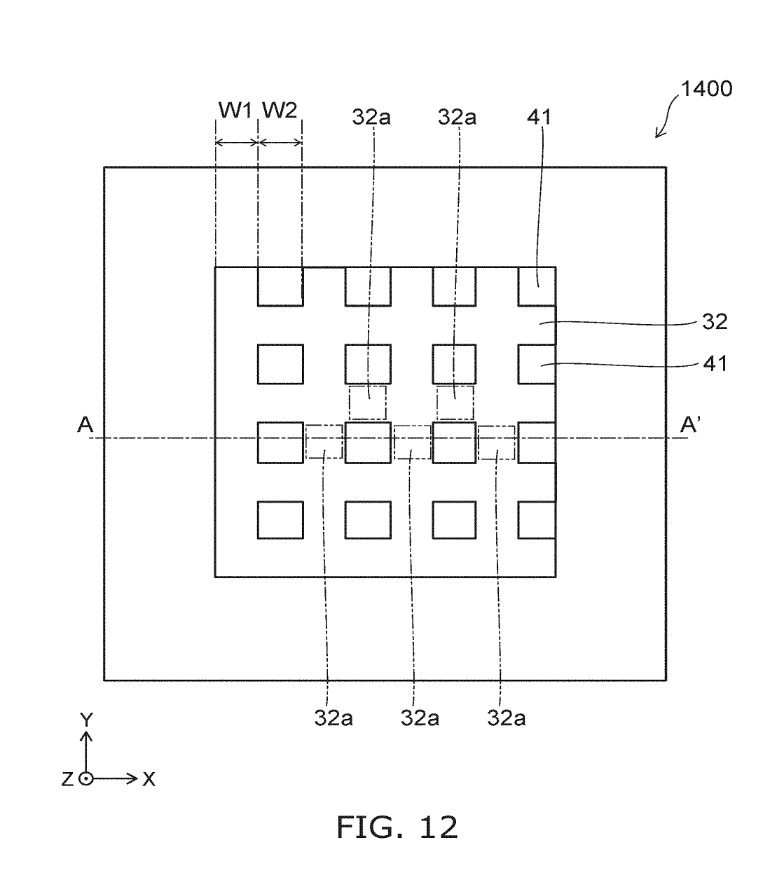

FIG. 14 is a schematic cross-sectional view illustrating another example of the detection device according to the second embodiment. As in the detection device 2100 illustrated in FIG. 14, a fifth layer 45 may be provided between the fourth electrode 34 and the photoelectric conversion layer 51; and a sixth layer 46 may be provided between the third electrode 33 and the photoelectric conversion layer 51.

For example, the fifth layer 45 functions as an electron blocking layer that obstructs the flow of electrons, or a hole extraction layer (a trap layer) that makes it easy for holes to flow. The fifth layer 45 may further function as an exciton blocking layer for confining the excitons generated by the photoelectric conversion layer 51. For example, it is favorable for the fifth layer 45 to include a hole-accepting material. For example, a triarylamine compound, a benzidine compound, a pyrazoline compound, a styrylamine compound, a hydrazone compound, a triphenylmethane compound, a carbazole compound, a thiophene compound, a phthalocyanine compound, a condensed aromatic compound, etc., may be used as the hole-accepting material. For example, a naphthalene derivative, an anthracene derivative, a tetracene derivative, a pentacene derivative, a pyrene derivative, a perylene derivative, etc., may be used as the condensed aromatic compound.

For example, the sixth layer 46 functions as a hole blocking layer that obstructs the flow of holes. The sixth layer 46 may further the function as an exciton blocking layer for confining the excitons generated by the photoelectric conversion layer 51. For example, it is favorable for the sixth layer 46 to include an electron-accepting material. For example, an oxadiazole derivative, a triazole compound, an anthraquinodimethane derivative, a diphenylquinone derivative, bathocuproine, a bathocuproine derivative, bathophenanthroline, a bathophenanthroline derivative, a 1,4,5,8-naphthalenetetracarboxylic diimide derivative, naphthalene-1,4,5,8-tetracarboxylic dianhydride, etc., may be used as the electron-accepting material.

In the detection device 2100, the function of the fifth layer 45 and the function of the sixth layer 46 may be reversed.

### Third Embodiment

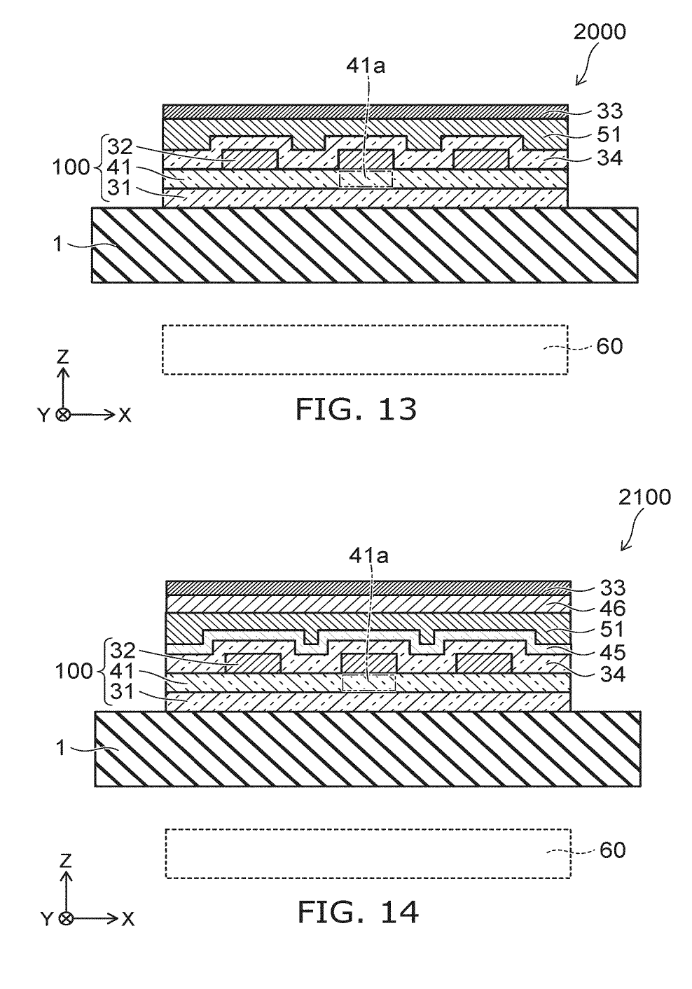

FIG. 15 is a schematic cross-sectional view illustrating an example of a detection device according to a third embodiment. In FIG. 15, an example of the optical path is illustrated by the broken line. The detection device 3000 includes, for example, the substrate 1, the light detector 50, the multiple second electrodes 32, the light-emitting layer 41, and the first electrode 31.

In the detection device 3000, the light detector 50 is provided between the first electrode 31 and at least a portion of the substrate 1. The light-emitting layer 41 is provided between the light detector 50 and the first electrode 31. The second electrodes 32 are provided between a portion of the light-emitting layer 41 and a portion of the light detector 50. For example, the second electrodes 32 are multiply provided in the second direction. For example, the structures illustrated in any of FIG. 9A to FIG. 12 also are employable as the structure of the second electrodes 32.

The light that is radiated from the light-emitting regions 41a of the light-emitting layer 41 passes through the first electrode 31 and is incident on the detection object 60. The information that relates to the detection object 60 can be obtained by the light being reflected by the detection object 60, passing between the second electrodes 32, and being incident on the light detector 50.

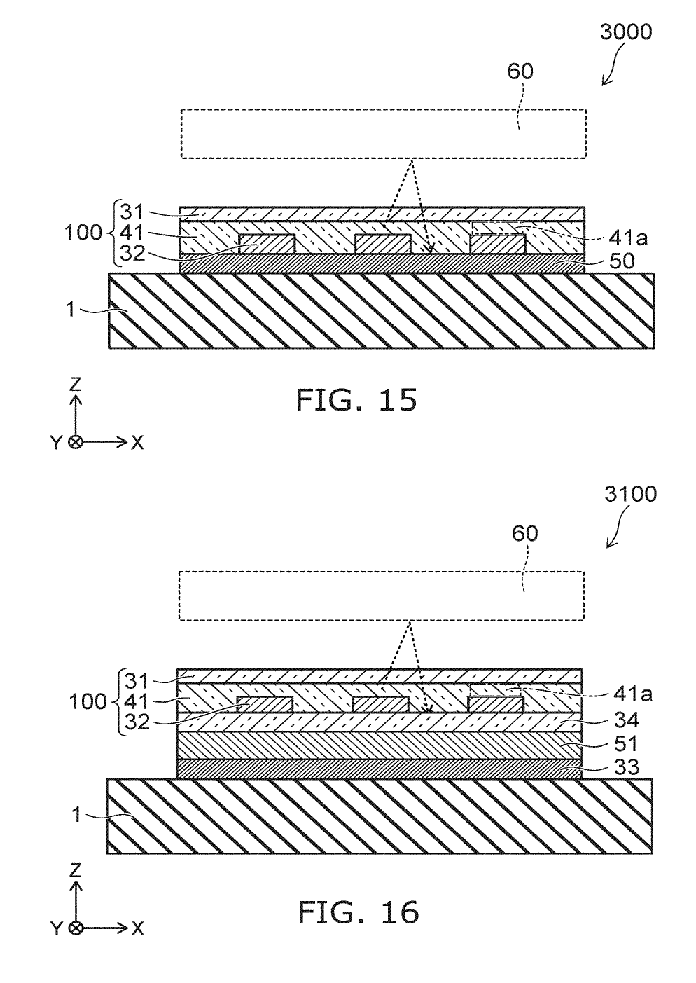

FIG. 16 is a schematic cross-sectional view illustrating another example of the detection device according to the third embodiment. As illustrated in FIG. 16, the detection device 3100 includes, for example, the substrate 1, the third electrode 33, the photoelectric conversion layer 51, the fourth electrode 34, the multiple second electrodes 32, the light-emitting layer 41, and the first electrode 31.

The light-emitting layer 41 is provided between the third electrode 33 and the fourth electrode 34. The fourth electrode 34 is provided between the photoelectric conversion layer 51 and the light-emitting layer 41. The fourth electrode 34 is light-transmissive. The multiple second electrodes 32 are provided between the fourth electrode 34 and the photoelectric conversion layer 51. The structures illustrated in any of FIG. 9A to FIG. 12 also are employable as the structure of the second electrodes 32.

### Fourth Embodiment

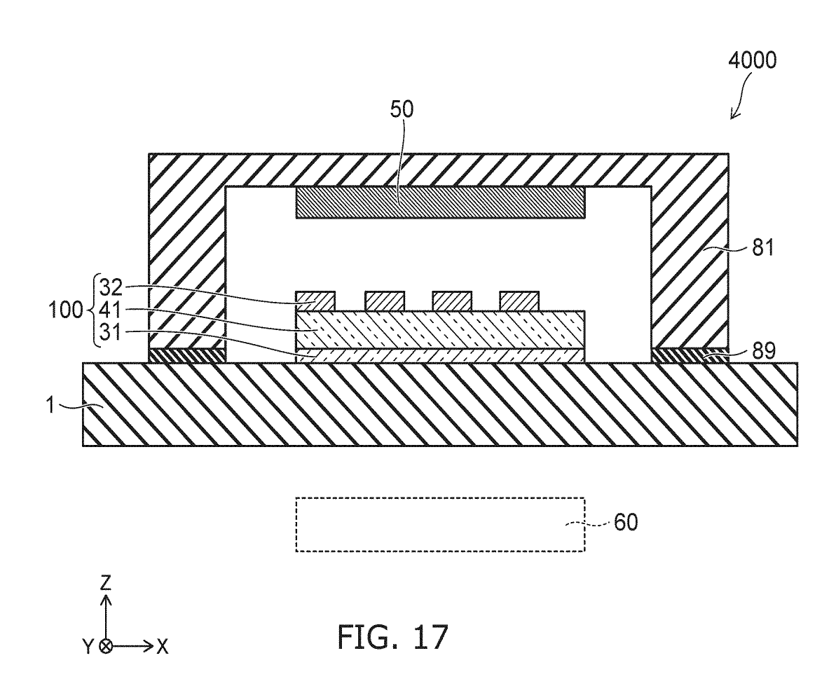

FIG. 17 is a schematic cross-sectional view illustrating an example of a detection device according to a fourth embodiment. The detection device 4000 further includes, for example, a sealing portion 81 in addition to the components included in the detection device 1000. The sealing portion 81 is provided to be separated from the light emitter 100 including the first electrode 31, the light-emitting layer 41, and the multiple second electrodes 32. The light emitter 100 is provided between the sealing portion 81 and the substrate 1 in the first direction and is surrounded with the sealing portion 81 along a plane perpendicular to the first direction.

For example, the sealing portion 81 includes glass and is bonded to the substrate 1 by a bonding agent 89. For example, nitrogen gas is filled into the interior of the sealing portion 81. For example, the light detector 50 is mounted to an inner wall of the sealing portion 81.

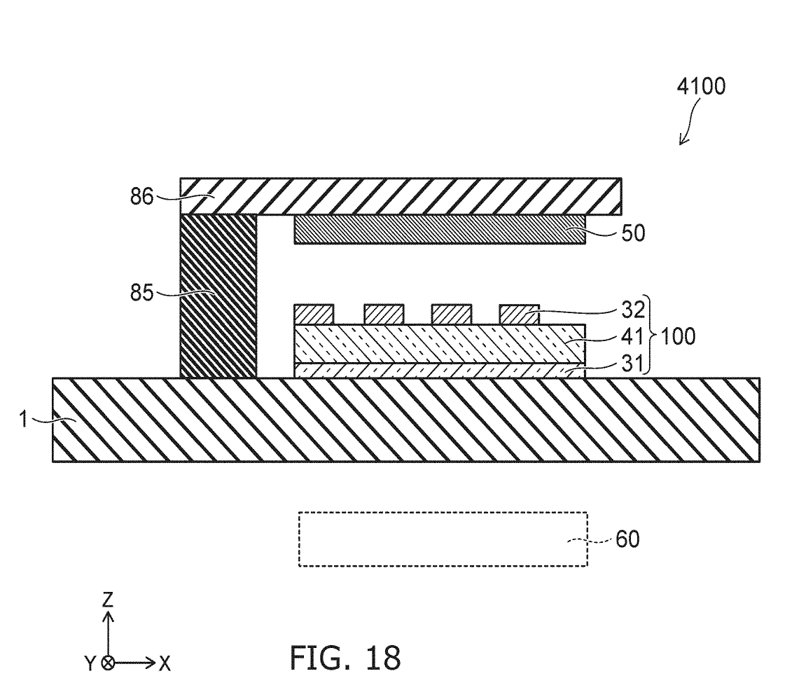

FIG. 18 is a schematic cross-sectional view illustrating another example of the detection device according to the fourth embodiment. The detection device 4100 includes the substrate 1, the light emitter 100, the light detector 50, a support portion 85, and a support platform 86. The support portion 85 is a member having a columnar configuration; and the support platform 86 is fixed to the substrate 1 via the support portion 85. The support portion 85 may be multiply provided around the light emitter 100. The light detector 50 is mounted to the support platform 86; and the light emitter 100 and the light detector 50 are positioned between the substrate 1 and the support platform 86.

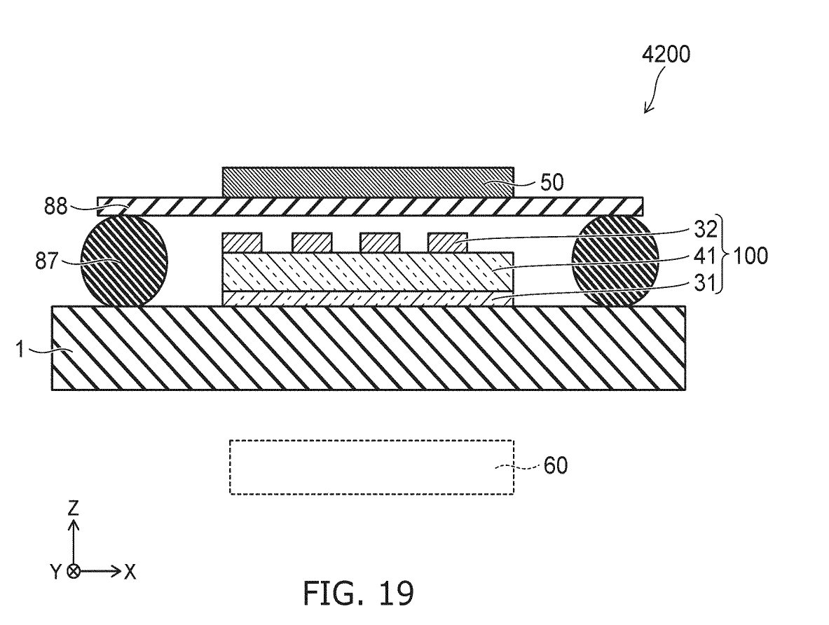

FIG. 19 is a schematic cross-sectional view illustrating another example of the detection device according to the fourth embodiment. The detection device 4200 includes the substrate 1, the light emitter 100, the light detector 50, a support portion 87, and a support plate 88. The support portion 87 is, for example, a member in which the cross section along a plane including the first direction and the third direction is a circle. The configuration of the cross section is arbitrary and may be a quadrilateral. For example, the support portion 87 is provided in an annular configuration along a plane perpendicular to the first direction on the substrate 1. The support plate 88 is fixed to the substrate 1 via the support portion 87. The support plate 88 is light-transmissive. The light detector 50 is provided on the support plate 88; and the support plate 88 is positioned between the light detector 50 and the light emitter 100.

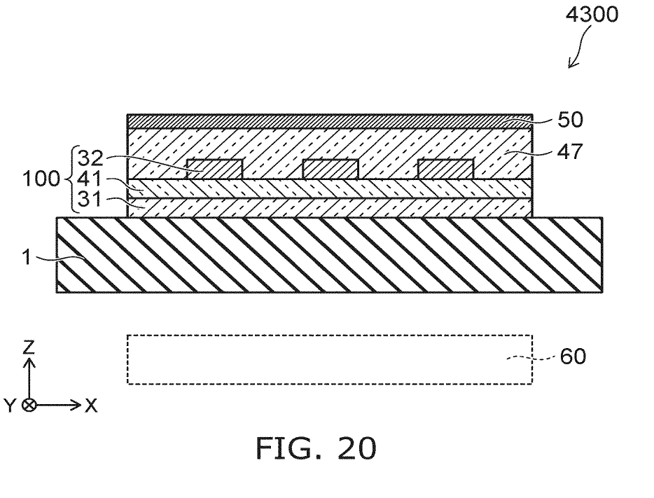

FIG. 20 is a schematic cross-sectional view illustrating another example of the detection device according to the fourth embodiment. The detection device 4300 includes the substrate 1, the light emitter 100, a seventh layer 47, and the light detector 50. The seventh layer 47 is provided between the light emitter 100 and the light detector 50. The seventh layer 47 is light-transmissive and includes an insulating material. The seventh layer 47 includes, for example, at least one of polyimide or silicon oxide (SiO₂).

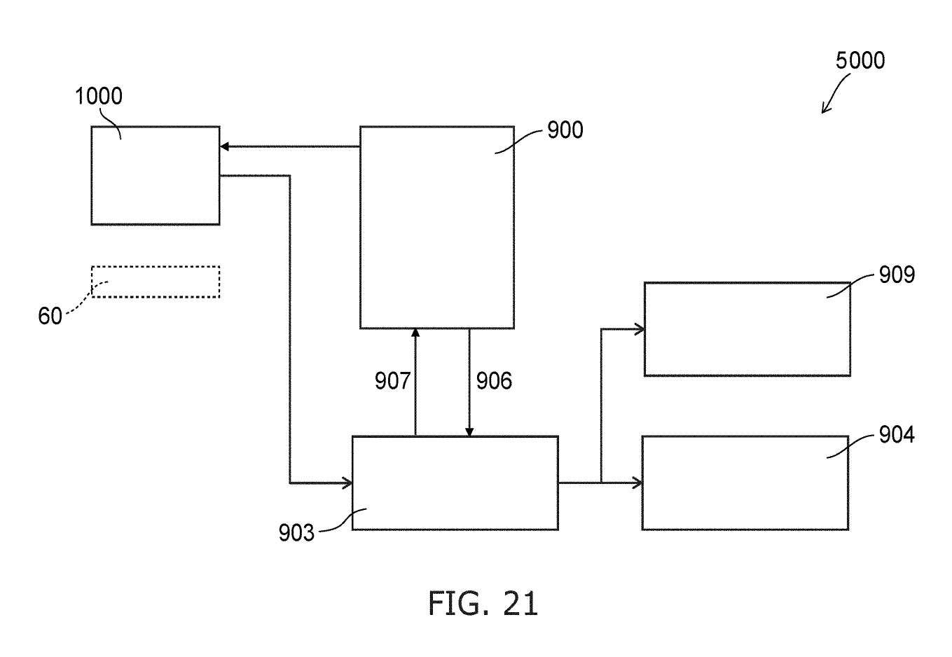

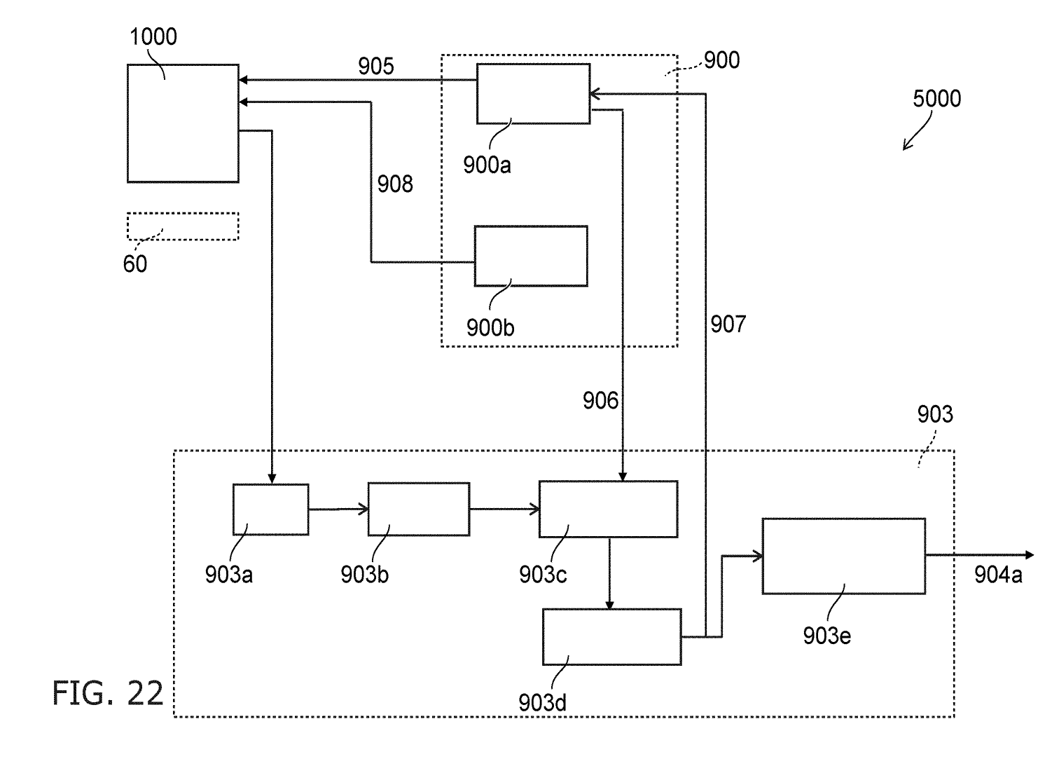

FIG. 21 and FIG. 22 are schematic views illustrating examples of a processing apparatus including the detection device according to the embodiment. As illustrated in FIG. 21, the processing apparatus 5000 includes, for example, the detection device 1000, a controller 900, a signal processor 903, a recording device 904, and a display device 909. The processing apparatus 5000 may include another detection device according to the embodiment instead of the detection device 1000.

The detection device 1000 that receives an input signal from the controller 900 emits light from the light emitter 100. The light that is emitted is reflected by the detection object 60 and is detected by the light detector 50 of the detection device 1000. The detection device 1000 may receive a bias signal from the controller 900 to increase the detection sensitivity of the light detector 50.

The signal that is detected by the light detector 50 is output to the signal processor 903. The signal processor 903 receives the signal from the detection device 1000 and performs, for example, processing of the signal such as AC detection, signal amplification, noise removal, etc., as appropriate. The signal processor 903 may receive a synchronization signal from the controller 900 to perform the appropriate signal processing. A feedback signal may be transmitted from the signal processor 903 to the controller 900 to adjust the light amount radiated from the light emitter 100. The signal that is generated by the signal processor 903 is stored in the recording device 904; and the information is displayed in the display device 909.

The processing apparatus 5000 may not include the recording device 904 and the display device 909. In such a case, the signal that is generated by the signal processor 903 is output to, for example, a recording device and a display device outside the processing apparatus 5000.

The processing apparatus 5000 will now be described more specifically with reference to FIG. 22. As illustrated in FIG. 22, the light emitter of the detection device 1000 receives an input signal 905 including a DC bias signal or a pulse signal from a pulse generator 900a of the controller 900. The light that is emitted from the light emitter 100 is reflected by the detection object 60 and detected by the light detector 50. The light detector 50 may receive a bias signal from a bias circuit 900b of the controller 900. The signal that is detected by the light detector 50 is input to the signal processor 903. After AC detection of the signal from the light detector is performed as necessary by the signal processor 903, the signal is amplified by an amplifier 903a; and unnecessary noise components are removed by a filter portion 903b. A signal synchronizer 903c receives the signal output from the filter portion 903b, and if appropriate, receives a synchronization signal 906 from the controller 900 and performs synchronization with the light.

The signal that is output from the signal synchronizer 903c is input to a signal shaper 903d. The processing apparatus 5000 may not include the signal synchronizer 903c. In such a case, the signal that is output from the filter portion 903b is input to the signal shaper 903d without going through the signal synchronizer 903c.

In the signal shaper 903d, the signal is shaped into the desired signal so that the appropriate signal processing is performed by a signal calculator 903e. For example, the signal shaping is performed by time averaging, etc. In the signal processor 903, the order of the AC detection and the processing performed by the processors is modifiable as appropriate. A calculated value 904a from the signal calculator 903e of the signal processor 903 is output to a recording device and a display device.

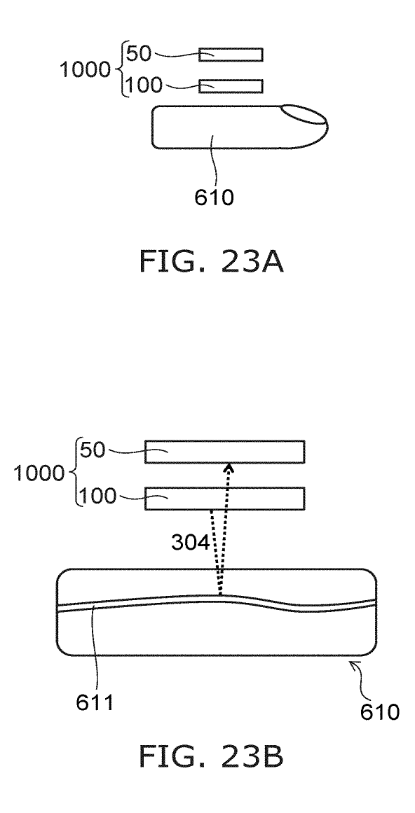

FIG. 23A to FIG. 26B are schematic views illustrating a pulse wave being measured using the detection device according to the embodiment. Although the detection device 1000 is used in the examples illustrated in FIG. 23A to FIG. 26B, another detection device according to the embodiment may be used instead of the detection device 1000.

FIG. 23A and FIG. 23B illustrate the detection of the pulse wave of a blood vessel 611 inside a finger 610. FIG. 23B is a schematic view of an enlarged portion of FIG. 23A. Other than the finger 610, the living body location may be selected arbitrarily to be an ear, a chest, an arm, etc. In the example illustrated in FIGS. 23A and 23B, light 304 that is emitted from the light emitter 100 is reflected by the blood vessel 611 and is detected by the light detector 50. At this time, the light detector 50 detects a signal reflecting the blood flow of the blood vessel 611. For example, the pulse is measured by the signal processor 903 illustrated in FIG. 21 and FIG. 22 performing signal processing of the signal that is detected.

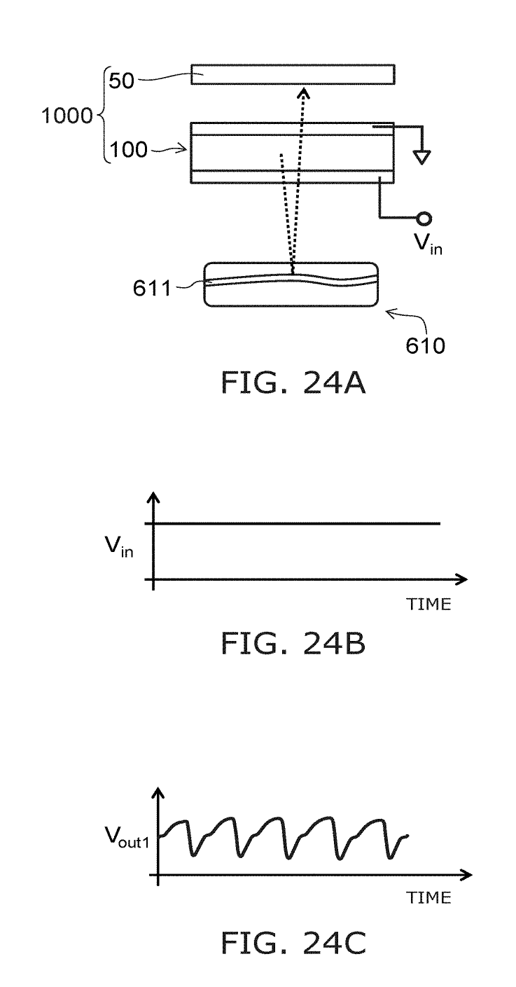

As illustrated in FIG. 24B, for example, a constant voltage is applied as an input signal Vᵢₙ to the first electrode 31 and the second electrodes 32 of the light emitter 100. As illustrated in FIG. 24A, the light detector 50 detects the light reflected by the finger 610. At this time, as illustrated in FIG. 24C, the signal inside the blood is superimposed onto a signal Vₒᵤₜ detected by the light detector 50.

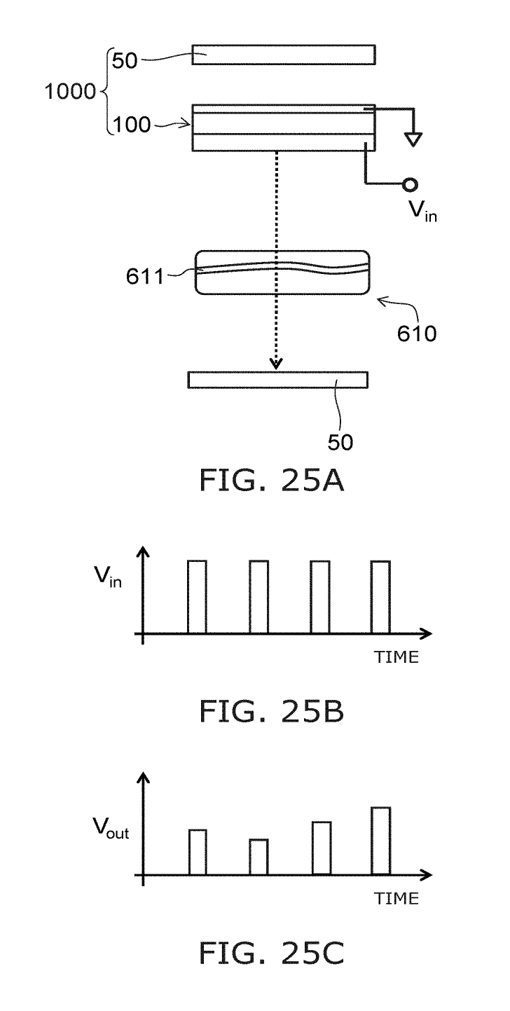

Or, as illustrated in FIG. 25A and FIG. 25B, the light may be radiated from the light emitter 100 by applying a pulse voltage as the input signal Vᵢₙ to the first electrode 31 and the second electrodes 32 of the light emitter 100. As illustrated in FIG. 25C, the light on which the signal inside the blood is superimposed is detected by the light detector 50.

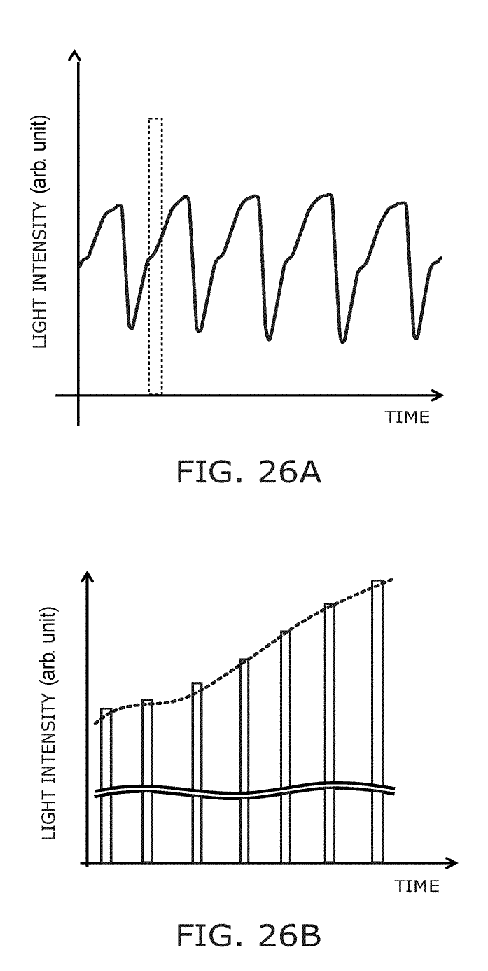

FIGS. 26A and 26B illustrate an example of the optical signal detected in the case where a pulse voltage is applied as the input signal Vᵢₙ. FIG. 26B illustrates the enlarged portion surrounded with the broken line of FIG. 26A. In the case where the frequency of the pulse voltage applied to the light emitter 100 is sufficiently faster than the frequency of the pulse wave, the pulse wave signal is obtained by viewing only the optical signal of each light pulse as illustrated in FIGS. 26A and 26B. Typically, the pulse wave is about 1 Hz; and the frequency of the pulse voltage may be set to, for example, 100 Hz to 100 KHz. Because the time that the light emitter 100 emits light is shorter for the configuration in which the pulse voltage illustrated in FIG. 25A to FIG. 26B is used than for the configuration in which the constant voltage illustrated in FIGS. 24A to 24C is used, this is advantageous in that the degradation of the light emitter 100 is suppressed; and the power consumption can be reduced.

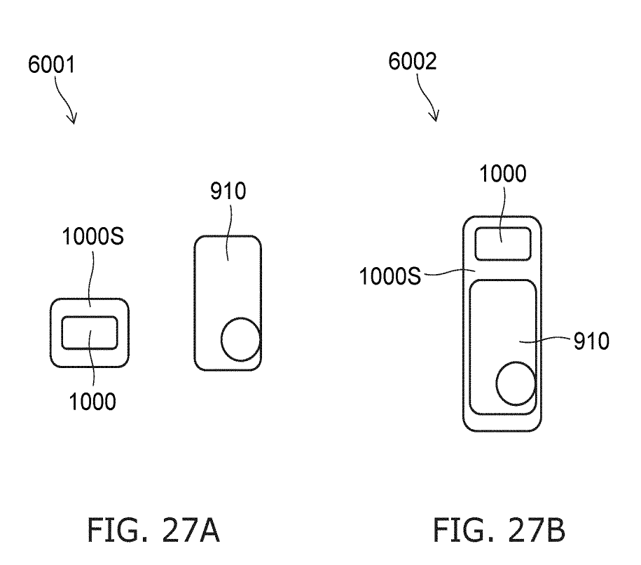

FIG. 27A and FIG. 27B are schematic views illustrating processing apparatuses including the detection device according to the embodiment. The processing apparatuses 6001 and 6002 include the detection device 1000 and a controller/signal processor 910. These processing apparatuses may include another detection device according to the embodiment instead of the detection device 1000.

In the processing apparatus 6001, the detection device 1000 is provided on a support substrate 1000S. The processing apparatus 6001 has a configuration in which the detection device 1000 and the controller/signal processor 910 are provided independently from each other.

In the processing apparatus 6002, the detection device 1000 and the controller/signal processor 910 are provided on a common support substrate 1000S.

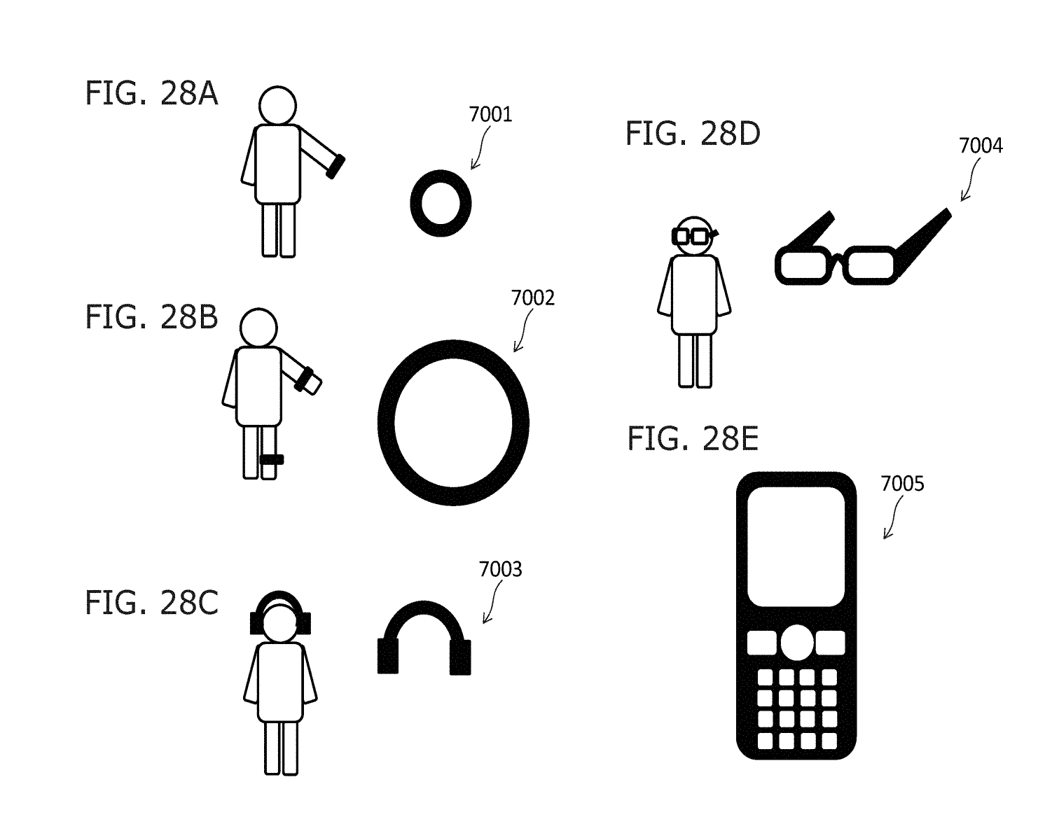

FIG. 28A to FIG. 28E are schematic views illustrating applications of processing apparatuses including the detection device according to the embodiment. The processing apparatus in each example measures, for example, the pulse and/or the oxygen concentration of blood.

In the example illustrated in FIG. 28A, a processing apparatus 7001 is included in a finger ring. For example, the processing apparatus 7001 detects the pulse of a finger contacting the processing apparatus 7001. In the example illustrated in FIG. 28B, a processing apparatus 7002 is included in an arm band. For example, the processing apparatus 7002 detects the pulse of an arm or a leg contacting the processing apparatus 7002.

In the example illustrated in FIG. 28C, a processing apparatus 7003 is included in an earphone. In the example illustrated in FIG. 28D, a processing apparatus 7004 is included in eyeglasses. For example, the processing apparatuses 7003 and 7004 detect the pulse of an ear lobe. In the example illustrated in FIG. 28E, a processing apparatus 7005 is included in a button, a screen, etc., of a mobile telephone or a smartphone. For example, the processing apparatus 7005 detects the pulse of a finger touching the processing apparatus 7005.

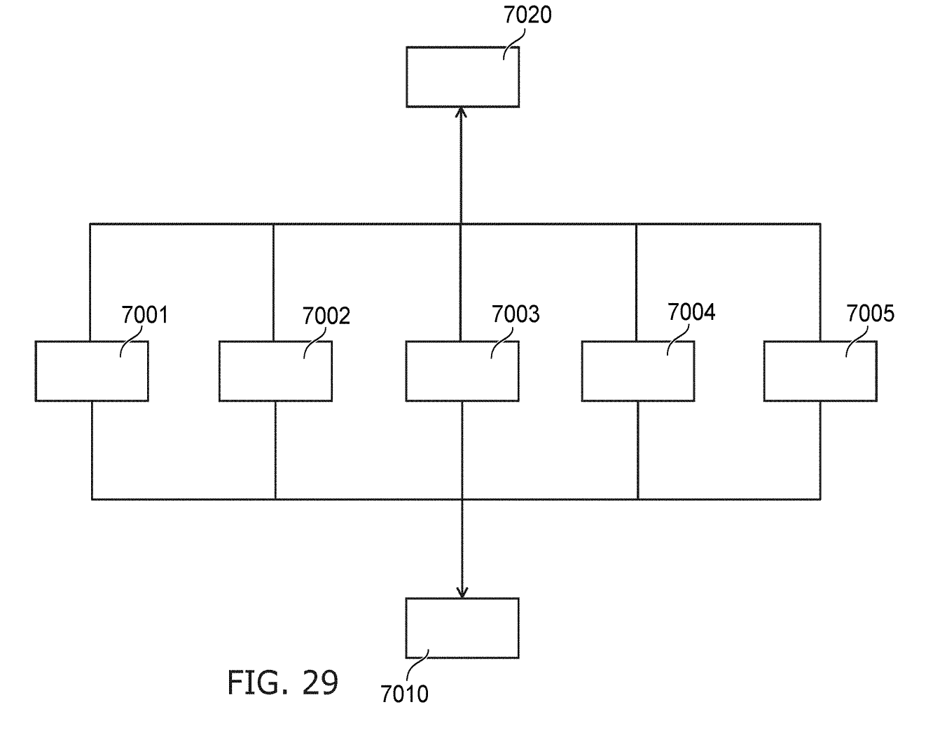

FIG. 29 is a schematic view illustrating a system including the processing apparatuses illustrated in FIGS. 28A to 28E.

For example, the processing apparatuses 7001 to 7005 transmit the measured data to a device 7010 such as a desktop PC, a notebook PC, a tablet terminal, etc., by a wired or wireless method. Or, the processing apparatuses 7001 to 7005 may transmit the data to a network 7020.

The data that is measured by the processing apparatuses can be monitored by utilizing the device 7010 or the network 7020. Or, monitoring or statistical processing may be performed by analyzing the measured data by using an analysis program, etc. In the case where the measured data is a pulse or an oxygen concentration of blood, the summary of the data may be performed at any time interval. For example, the data that is summarized is utilized for health care. At a hospital, for example, the data is utilized for continuous monitoring of the health condition of a patient.

According to the embodiments recited above, a detection device and a processing apparatus can be provided in which a smaller size is possible.

In the specification of the application, “perpendicular” and “parallel” refer to not only strictly perpendicular and strictly parallel but also include, for example, the fluctuation due to manufacturing processes, etc. It is sufficient to be substantially perpendicular and substantially parallel.

Hereinabove, embodiments of the invention are described with reference to specific examples. However, the invention is not limited to these specific examples. For example, one skilled in the art may similarly practice the invention by appropriately selecting specific configurations of components included in the detection device and the processing apparatus such as the substrate 1, the first electrode 31, the third electrode 33, the fourth electrode 34, the light-emitting layer 41, the third layer 43, the fourth layer 44, the fifth layer 45, the sixth layer 46, the seventh layer 47, the light detector 50, the photoelectric conversion layer 51, the sealing portion 81, the controller 900, the signal processor 903, the recording device 904, and the display device 909, etc., from known art; and such practice is within the scope of the invention to the extent that similar effects can be obtained.

Further, any two or more components of the specific examples may be combined within the extent of technical feasibility and are included in the scope of the invention to the extent that the purport of the invention is included.

Moreover, all the detection devices and the processing apparatuses practicable by an appropriate design modification by one skilled in the art based on the detection devices and the processing apparatuses described above as embodiments of the invention also are within the scope of the invention to the extent that the spirit of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A detection device, comprising:
a substrate, the substrate being light-transmissive;
a light detector; and
a light emitter provided between the substrate and the light detector, the light emitter including
a first electrode provided between the light detector and the substrate, the first electrode being light-transmissive,
a light-emitting layer provided between the light detector and the first electrode, and
a plurality of second electrodes provided between the light detector and the light-emitting layer.

2. The device according to claim 1, wherein the plurality of second electrodes is arranged in a second direction perpendicular to a first direction, the first direction being from the substrate toward the light detector.

3. The device according to claim 2, wherein a length in a third direction of the second electrode is longer than a length in the second direction of the second electrode, the third direction being perpendicular to the first direction and the second direction.

4. The device according to claim 1, wherein a reflectance of the second electrode is higher than a reflectance of the first electrode.

5. The device according to claim 1, wherein the light-emitting layer includes an organic substance.

6. The device according to claim 1, wherein
the light detector includes:
a third electrode;
a fourth electrode provided between the light emitter and the third electrode, the fourth electrode being light-transmissive; and
a photoelectric conversion layer provided between the third electrode and the fourth electrode.

7. The device according to claim 6, wherein a portion of the fourth electrode is provided between the second electrodes in a second direction perpendicular to a first direction, the first direction being from the substrate toward the light detector.

8. The device according to claim 6, wherein an injection barrier between the fourth electrode and the light-emitting layer is larger than an injection barrier between the second electrode and the light-emitting layer.

9. The device according to claim 1, further comprising a carrier injection layer,
at least a portion of the carrier injection layer being provided between the light-emitting layer and at least one of the plurality of second electrodes.

10. The device according to claim 1, further comprising a sealing portion,
the light-emitting layer being provided between a portion of the sealing portion and a portion of the substrate in the first direction,
the light-emitting layer being surrounded with the sealing portion along a plane perpendicular to the first direction.

11. A detection device, comprising:
a substrate;
a first electrode, the first electrode being light-transmissive;
a light detector provided between the substrate and the first electrode;
a light-emitting layer provided between the light detector and the first electrode; and
a plurality of second electrodes provided between the light detector and the light-emitting layer.

12. The device according to claim 11, wherein the plurality of second electrodes is arranged in a second direction perpendicular to a first direction, the first direction being from the substrate toward the light detector.

13. The device according to claim 12, wherein a length in a third direction of the second electrode is longer than a length in the second direction of the second electrode, the third direction being perpendicular to the first direction and the second direction.

14. The device according to claim 11, wherein
the light detector includes:
a third electrode;
a fourth electrode provided between the first electrode and the third electrode, the fourth electrode being light-transmissive; and
a photoelectric conversion layer provided between the third electrode and the fourth electrode.

15. The device according to claim 14, wherein an injection barrier between the fourth electrode and the light-emitting layer is larger than an injection barrier between the second electrode and the light-emitting layer.

16. The device according to claim 11, further comprising a carrier injection layer,
at least a portion of the carrier injection layer being provided between the light-emitting layer and at least one of the plurality of second electrodes.

17. A detection device, comprising:
a substrate, the substrate being light-transmissive;
a light detector; and
a light emitter provided between the substrate and the light detector, the light emitter including
a first electrode provided between the light detector and the substrate, the first electrode being light-transmissive,
a light-emitting layer provided between the light detector and the first electrode, and
a second electrode provided between a portion of the light detector and a portion of the light-emitting layer,
the second electrode including a plurality of first portions provided to be separated from each other.

18. The device according to claim 17, wherein the plurality of first portions is arranged in a second direction perpendicular to a first direction, the first direction being from the substrate toward the light detector.

19. The device according to claim 17, wherein
the light detector includes:
a third electrode;
a fourth electrode provided between the light emitter and the third electrode, the fourth electrode being light-transmissive; and
a photoelectric conversion layer provided between the third electrode and the fourth electrode.

20. A processing apparatus, comprising:
the detection device according to claim 1; and
a processor receiving and processing a signal detected by the detection device.
